(19) United States
(12) Patent Application Publication
(10) Pub. No.: US 2023/0063364 A1
(43) Pub. Date: Mar. 2, 2023

(54) ULTRASOUND-CONTROLLED TRAINING PROGRAM FOR INDIVIDUALIZED AND AUTOMATIC WEANING

(71) Applicant: Koninklijke Philips N.V., Eindhoven (NL)

(72) Inventors: Cornelis Petrus Hendriks, Eindhoven (NL); Roberto Buizza, Eindhoven (NL); Kiran Hamilton J. Dellimore, Eindhovven (NL); Michael Polkey, Monroeville, PA (US); Jaap Roger Haartsen, Eindhoven (NL); Joerg Sabczynski, Norderstedt (DE); Thomas Koehler, Hamburg (DE); Nataly Wieberneit, Hamburg (DE); Rafael Wiemker, Kisdorf (DE); Rita Priori, Utrecth (NL)

(21) Appl. No.: 17/857,484

(22) Filed: Jul. 5, 2022

Related U.S. Application Data
(60) Provisional application No. 63/238,484, filed on Aug. 30, 2021.

(51) Int. Cl. A61M 16/00

(52) U.S. Cl. CPC A61M 16/024; A61M 16/0003; A61M 16/0051; A61M 2016/0027

(57) ABSTRACT

A mechanical ventilation device comprises at least one electronic controller configured to: receive ultrasound data related to a thickness of a diaphragm of a patient during inspiration and expiration while the patient undergoes mechanical ventilation therapy with a mechanical ventilator; calculate a diaphragm thickness metric based on at least the ultrasound data; and when the calculated diaphragm thickness metric does not satisfy an acceptance criterion, at least one of: output an alert indicative of the calculated diaphragm thickness metric failing to satisfy the acceptance criterion; and output a recommended adjustment to one or more parameters of the mechanical ventilation therapy delivered to the patient.

### CROSS-REFERENCE TO RELATED APPLICATIONS

This patent application claims the priority benefit under 35 U.S.C. § 119(e) of U.S. Provisional Application No. 63/238,484, filed on Aug. 30, 2021, the contents of which are herein incorporated by reference.

The following relates generally to the respiratory therapy arts, mechanical ventilation arts, mechanical ventilation weaning arts, and related arts.

### BACKGROUND

An important consideration in ventilation therapy is that a patient does not remain on mechanical ventilation (MV) for longer than necessary, since prolonged mechanical ventilation is associated with many risks such as pneumonia, poor long-term functional outcomes, and higher healthcare costs. A limiting factor for successful weaning is ventilator-induced diaphragm dysfunction (VIDD), in which dysfunction of the diaphragm is caused by muscle fiber injury and atrophy. The underlying cause is thought to be due in large part to the ventilation therapy itself since disuse of muscles is known to cause muscle atrophy, although other clinical features which are common in the critically ill (e.g., inflammation) may also contribute to VIDD. Ideally these changes in diaphragm structure and function should be minimized or prevented, if possible, for example by early detection and the introduction of training stimuli.

Most patients (i.e., approximately 60-70%) require minimal or no weaning of ventilatory support and are extubated without difficulty after the first spontaneous breathing trial (SBT). These patients may be classified as simple weaning. The remaining 30-40% of the patients may be classified as difficult weaning. These patients with difficult weaning would benefit from an improved weaning process. Additionally, from the health economics perspective it is beneficial to address high-cost patients, since these patients utilized almost 40% of the ICU cost although they constitute only about 10% of the ICU patients (see, e.g., Aung Y. N. et al., 2019, “Characteristics and outcome of high-cost ICU patients”, ClinicoEconomics and Outcomes Research 2019:11 505-513).

Current weaning strategies include timely recognizing the readiness to wean and the readiness to extubate (see, e.g., Rose, L., 2015, “Strategies for weaning from mechanical ventilation: A state of the art review”, Intensive and Critical Care Nursing, Vol 31 (4), Pages 189-195). A spontaneous breathing trial (SBT) currently is advocated as the best method to assess extubation readiness. The respiratory therapist (RT) starts an SBT and observes the patient's response. As noted, approximately 60-70%, of patients require minimal or no weaning of ventilatory support and are extubated without difficulty but the remaining 30-40% require a more graduated approach to reducing the amount of support provided by the ventilator. The challenge is to balance reloading and the prevention of overloading of the diaphragm muscle (that is, to balance returning the breathing effort to the diaphragm without overloading the diaphragm which can lead to muscle fatigue, damage, or so forth). Guidelines prescribe the use of pressure support ventilation (PSV) or intermittent SBTs to reload the weak diaphragm gradually or intermittently. However, animal studies have shown that PSV or intermittent SBT can even further decrease the diaphragm force (see, e.g., Bruells et al., 2016, “Influence of weaning methods on the diaphragm after mechanical ventilation in a rat model”, BMC Pulmonary Medicine (2016) 16:127, in which close monitoring of the diaphragm, for example the trans-diaphragm pressure or its electrical activity, is used to guide weaning individually.

For difficult to wean patients, automated weaning systems and ventilator modes that promote improved patient-ventilator interaction can be used. Automated weaning systems adapt the ventilatory support to the patients through continuous monitoring and real-time intervention. An example is the SmartCare/PS system (available from Draeger, Luebeck, Germany) which adapts the pressure, waits for the patient to become stable, adapts the pressure again, and so forth until the pressure support is reduced to almost zero. Such automated weaning systems can be viewed as a computerized version of a written weaning protocol.

Systems with a more sophisticated assisted mode make use of observing the patient-ventilator interaction. They measure the respiratory effort, and they provide a pressure that is proportional to the respiratory effort. For example, in proportional assist ventilation (PAV or PAV+), the amount of assistance provided by the ventilator is automatically adjusted and proportional to the patient's effort, measured via the respiratory compliance and resistance throughout the inspiratory cycle. Therefore, pressure assistance adapts on a breath-by-breath basis to the patient's needs. The degree of assistance is set by the percentage-support setting (see, e.g., Kondili, E., et al., 2006, “Respiratory load compensation during mechanical ventilation—proportional assist ventilation with load-adjustable gain factors versus pressure support”, Intensive Care Med (2006) 32:692-699).

With neurally-adjusted ventilatory assist (NAVA) ventilation process, pressure delivered to the airway is proportional to inspiratory diaphragmatic electrical activity measured via an esophageal catheter. As with PAV, NAVA has been shown to reduce over-assistance provided by the ventilator and improve patient-ventilator interaction.

Ultrasound imaging for diaphragm function evaluation is receiving increasing attention because it is a simple, widely available bedside technique. For example, with ultrasound it is possible to detect diaphragm thickness, atrophy or recovery from atrophy, force and velocity of contraction, special patterns of motion, excursion, and changes in thickness during inspiration (see, e.g., Vivier et al., 2020, “Bedside Ultrasound for Weaning from Mechanical Ventilation”, Anesthesiology 2020; 132:947-8; Spiesshofer et al., 2020, “Evaluation of Respiratory Muscle Strength and Diaphragm Ultrasound: Normative Values, Theoretical Considerations, and Practical Recommendations”, Respiration 2020; 99:369-381; Matamis et al., 2013, “Sonographic evaluation of the diaphragm in critically ill patients. Technique and clinical applications”, Intensive Care Med (2013) 39:801-810; Tuinman et al., 2020, “Respiratory muscle ultrasonography: methodology, basic and advanced principles and clinical applications in ICU and ED patients—a narrative review”, Intensive Care Med (2020) 46:594-605). The ultrasound is a diagnostic complement to clinical examination, for example, to support a differential diagnosis of weaning failure.

Ultrasound imaging can be used for diaphragm-protective mechanical ventilation during mechanical ventilation, i.e., to titrate the ventilator support between over- and under-assistance. A criterium for safe physiological limits, leading to a stable muscle thickness, is to keep the diaphragm thickening fraction (TFdi) between 15 and 30%. NB. The TFdi is defined as the percentage increase in diaphragm thickness relative to end-expiratory thickness during tidal breathing. Diaphragm-protective mechanical ventilation may reduce likelihood of developing diaphragm atrophy thus making the patient more amenable to simple weaning, but does not assist in cases in which the patient experiences difficulties with a weaning process.

Diaphragm ultrasound imaging has also been used as an indicator of respiratory effort in post-operative patients undergoing assisted spontaneous breathing (see, e.g., Umbrello et al., 2015, “Diaphragm ultrasound as indicator of respiratory effort in critically ill patients undergoing assisted mechanical ventilation: a pilot clinical study”, Critical Care (2015) 19:161). Diaphragm thickening fraction was found to be a good indicator of changes of inspiratory muscle effort in response to modifications of the pressure support (PS) level.

The following discloses certain improvements to overcome these problems and others.

### SUMMARY

In one aspect, a mechanical ventilation device comprises at least one electronic controller configured to: receive ultrasound data related to a thickness of a diaphragm of a patient during inspiration and expiration while the patient undergoes mechanical ventilation therapy with a mechanical ventilator; calculate a diaphragm thickness metric based on at least the ultrasound data; and when the calculated diaphragm thickness metric does not satisfy an acceptance criterion, at least one of: output an alert indicative of the calculated diaphragm thickness metric failing to satisfy the acceptance criterion; and output a recommended adjustment to one or more parameters of the mechanical ventilation therapy delivered to the patient.

In another aspect, a mechanical ventilation method comprises, with at least one electronic controller: receiving ultrasound data related to a thickness of a diaphragm of patient during inspiration and expiration while the patient undergoes mechanical ventilation therapy with a mechanical ventilator; calculating a diaphragm thickness metric based on at least the ultrasound data; and when the calculated diaphragm thickness metric does not satisfy an acceptance criterion, at least one of: outputting an alert indicative of the calculated diaphragm thickness metric failing to satisfy the acceptance criterion; and outputting a recommended adjustment to one or more parameters of the mechanical ventilation therapy delivered to the patient.

One advantage resides in facilitating the weaning of patients off of mechanical ventilation therapy.

Another advantage resides in providing feedback control of a mechanical ventilation system based on feedback from an ultrasound system that monitors a diaphragm muscle response of a patient.

Another advantage resides in automatically adjusting settings of a mechanical ventilator to help wean patients off mechanical ventilation therapy.

Another advantage resides in providing mechanical ventilation therapy without the use of invasive catheters or dedicated ventilation maneuvers for measuring respiratory mechanics.

Another advantage resides in using a detected thickening fraction of the diaphragm to wean a patient off of mechanical ventilation therapy.

Another advantage resides in a controlled muscle training and response measurement, thereby providing a “diaphragm protective” method.

Another advantage resides in using ultrasound to non-invasively measure a diaphragm response.

Another advantage resides in using ultrasound to measure a diaphragm response independent of patient effort.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative mechanical ventilation system in accordance with the present disclosure.

FIGS. 2-4 show example flow charts of operations suitably performed by the system of FIG. 1.

### DETAILED DESCRIPTION

As used herein, the singular form of “a”, “an”, and “the” include plural references unless the context clearly dictates otherwise. As used herein, statements that two or more parts or components are “coupled,” “connected,” or “engaged” shall mean that the parts are j oined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

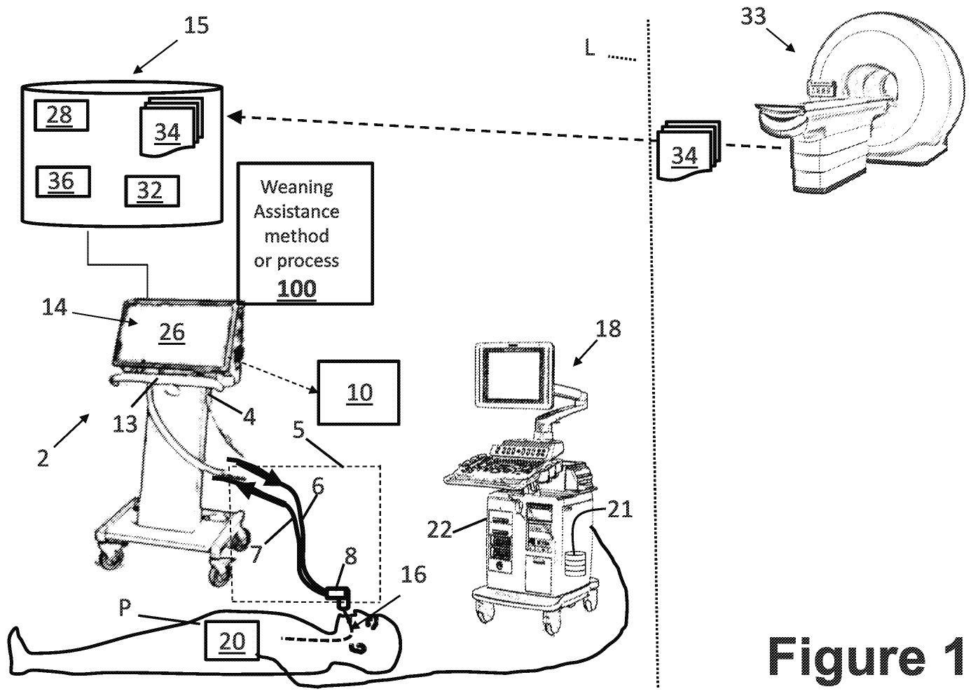

With reference to FIG. 1, a mechanical ventilator 2 for providing ventilation therapy to an associated patient P is shown. As shown in FIG. 1, the mechanical ventilator 2 includes an outlet 4 connectable with a patient breathing circuit 5 to delivery mechanical ventilation to the patient P. The patient breathing circuit 5 includes typical components for a mechanical ventilator, such as an inlet line 6, an optional outlet line 7 (this may be omitted if the ventilator employs a single-limb patient circuit), a connector or port 8 for connecting with an endotracheal tube (ETT) 16, and one or more breathing sensors (not shown), such as a gas flow meter, a pressure sensor, end-tidal carbon dioxide (etCO₂) sensor, and/or so forth. The mechanical ventilator 2 is designed to deliver air, an air-oxygen mixture, or other breathable gas (supply not shown) to the outlet 4 at a programmed pressure and/or flow rate to ventilate the patient via an ETT. The mechanical ventilator 2 also includes an electronic controller 13 (e.g., an electronic processor or a microprocessor), a display device 14, and a non-transitory computer readable medium 15 storing instructions executable by the electronic controller 13.

FIG. 1 diagrammatically illustrates the patient P intubated with an ETT 16 (the lower portion of which is inside the patient P and hence is shown in phantom). The connector or port 8 connects with the ETT 16 to operatively connect the mechanical ventilator 2 to deliver breathable air to the patient P via the ETT 16. The mechanical ventilation provided by the mechanical ventilator 2 via the ETT 16 may be therapeutic for a wide range of conditions, such as various types of pulmonary conditions like emphysema or pneumonia, viral or bacterial infections impacting respiration such as a COVID-19 infection or severe influenza, cardiovascular conditions in which the patient P receives breathable gas enriched with oxygen, or so forth.

FIG. 1 shows the patient P already intubated. That is, FIG. 1 shows the patient after a tracheal intubation has been performed to insert the ETT 16 into the patient. However, to safely perform the tracheal intubation, the anesthesiologist or other qualified medical professional first performs an assessment of the patient P to select the ETT size of the ETT 16, and then inserts an ETT of the selected size into the patient P by a tracheal intubation procedure.

FIG. 1 also shows a medical imaging device 18 (also referred to as an image acquisition device, imaging device, and so forth). As primarily described herein, the medical imaging device 18 comprises an ultrasound (US) medical imaging device 18. In other embodiments, the image acquisition device 18 can be a Computed Tomography (CT) image acquisition device, a C-arm imager, or other X-ray imaging device; Magnetic Resonance (MR) image acquisition device; or a medical imaging device of another modality. As described herein, the medical imaging device 18 is used to acquire images of the patient P. In some embodiments, the medical imaging device 18 can comprise a wearable US imaging device 18.

In a more particular example, the medical imaging device 18 includes an ultrasound transducer 20 that is wearable by the patient P (e.g., on the abdomen or chest of the patient P in position to image the diaphragm of the patient, as shown in FIG. 1). The US transducer 20 is positioned to acquire US imaging data (i.e., US images) of the diaphragm of the patient P. For example, the US transducer 20 is configured to acquire imaging data of a diaphragm of the patient P, and more particularly US imaging data related to a thickness of the diaphragm of a patient P during inspiration and expiration while the patient P undergoes mechanical ventilation therapy with the mechanical ventilator 2. The imaging device 18 also includes an electronic controller (e.g., an electronic processor or a microprocessor) 22 configured to receive the US imaging data from the US transducer 20. The imaging device 18 further includes a non-transitory storage medium 21 storing instructions executable by the electronic controller 22 to perform a mechanical ventilation weaning assistance method or process 100 for weaning the patient P off from mechanical ventilation therapy using the mechanical ventilator 2.

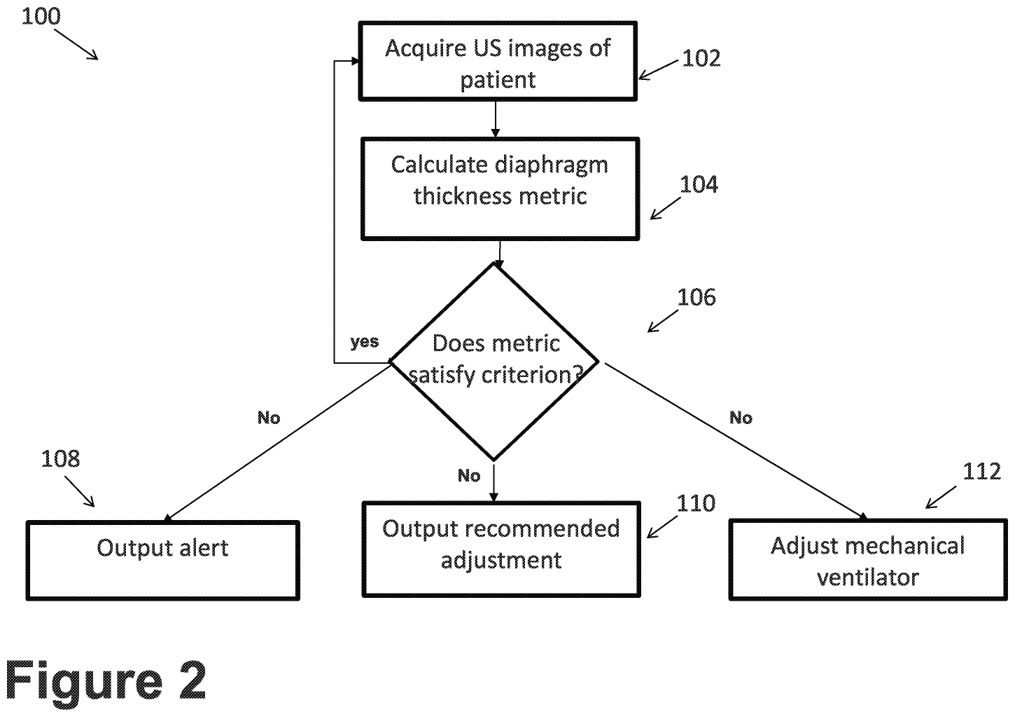

With reference to FIG. 2, and with continuing reference to FIG. 1, an illustrative embodiment of the weaning method 100 is diagrammatically shown as a flowchart. At an operation 102, the US imaging data is acquired by the US transducer 20, and is received by the electronic controller 22. The US imaging data can include data related to the respiratory effort by the patient P, including, for example, airway pressure, airway flow, and so forth.

At an operation 104, the electronic controller 22 is configured to calculate a diaphragm thickness metric based on (at least) the ultrasound data. In some embodiments, the diaphragm thickness metric includes a diaphragm thickening ratio (or fraction) TFdi indicative of a diaphragm thickness during inspiration relative to a diaphragm thickness during expiration. In a particular example, the diaphragm thickness metric includes a mean diaphragm thickness over a respiratory cycle. In another particular example, the diaphragm thickness metric includes a ratio of the maximum diaphragm thickness to the minimum diaphragm thickness over a breathing cycle. Optionally, the maximum diaphragm thickness used in calculating the ratio may be the average maximum diaphragm thickness over a sliding window spanning several breath cycles, and similarly for the minimum diaphragm thickness, in order to reduce noise. Typically, onset and/or progression of diaphragm atrophy is detected as a decrease in the diaphragm thickness over time.

At an operation 106, the electronic controller 22 is configured to determine whether the calculated diaphragm thickness metric satisfies a predetermined acceptance criterion. For example, if the medical imaging device 18 comprises a wearable US device 18 that acquires images of additional activities from respiratory muscles (i.e., an auxiliary muscle), the controller 13 can analyze the US images and the calculated diaphragm thickness metric to determine an effort by the patient P. If the electronic controller 22 determines that the calculated diaphragm thickness metric does not satisfy the predetermined acceptance criterion, the method 100 proceeds in one or more different ways. In one example embodiment, at an operation 108, an alert 26 indicative of the calculated diaphragm thickness metric failing to satisfy the acceptance criterion is output. The acceptance criterion may be, for example, that the diaphragm thickness metric exceeds a threshold value, as dropping below that threshold is considered to be an indication of onset of diaphragm atrophy. This alert output can be done by displaying a message on the display device 14 of the mechanical ventilator, or on a display of an electronic processing device (shown schematically in FIG. 1 as element 10), thereby indicating to a medical professional that the calculated diaphragm thickness metric is not satisfactory.

In another example embodiment, at an operation 110, a recommended adjustment to one or more parameters of the mechanical ventilation therapy delivered to the patient P is output. Again, this can be done by displaying a message on the display device 14 of the mechanical ventilator, or on the display of an electronic processing device 10, thereby indicating to a medical professional that the calculated diaphragm thickness metric is not satisfactory.

In a further example embodiment, at an operation 112, the mechanical ventilator 2 is controlled to adjust one or more parameters of the mechanical ventilation therapy delivered to the patient P. It will be appreciated that more than one of the operations 108, 110, and 112 can be performed (e.g., the alert 26 can be displayed and the settings of the mechanical ventilator 2 can be adjusted). In some embodiments, the operations 102-106 and at least one of operations 108-112 can be repeated iteratively to provide feedback control of the mechanical ventilator 2 based at least on whether the calculated diaphragm thickness metric satisfies the acceptance criterion.

In other embodiments, the electronic controller 13 of the mechanical ventilator 2 is configured to perform the method 100 (i.e., in lieu of the electronic controller 22 of the medical imaging device 18).

In such embodiments, the mechanical ventilation therapy delivered to the patient P comprises a mechanical ventilation training program. In such embodiments, when the calculated diaphragm thickness metric does not satisfy the acceptance criterion (i.e., the determination operation 106), the mechanical ventilation training program is adjusted until the calculated diaphragm thickness metric satisfies the acceptance criterion.

In some examples, the calculated diaphragm thickness metric comprises a respiratory muscle pressure Pₘᵤₛ calculated from the ultrasound data and a biomechanical model 28 (stored in non-transitory computer readable medium 15 of the mechanical ventilator 2). In this case, diaphragm atrophy is indicated as an undesirably low value for the calculated respiratory muscle pressure Pₘᵤₛ indicating the patient's diaphragm is unable to produce a satisfactory level of respiratory effort. The electronic controller 13 is configured to adjust the mechanical ventilation training program until the calculated respiratory muscle pressure Pₘᵤₛ satisfies the acceptance criterion by adjusting a level-of-support parameter of the mechanical ventilation training program. For example, in proportional assist ventilation (PAV or PAV+), the degree of assistance is set by the percentage level-of-support parameter K which scales the airway pressure (P_{aw}) delivered to the patient, i.e.:

P_{aw}(at level-of-support)=K×P_{aw}(full-support)

See e.g., Kondili, E., et al., 2006, “Respiratory load compensation during mechanical ventilation—proportional assist ventilation with load-adjustable gain factors versus pressure support”, Intensive Care Med (2006) 32:692-699. To adjust the mechanical ventilation training program, the electronic controller 13 is configured to multiply the level-of-support parameter by the respiratory muscle pressure Pₘᵤₛ to determine an airway ventilation pressure value, and continuously perform the mechanical ventilation training program until the airway ventilation pressure value falls below a predetermined training program threshold.

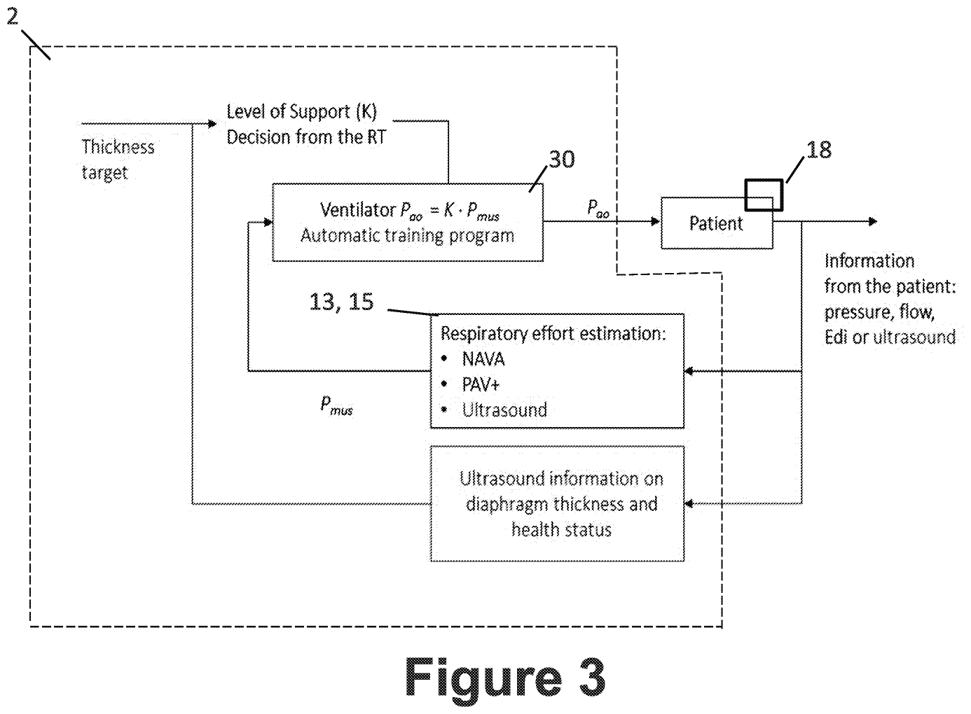

With continuing reference to FIGS. 1 and 2, FIG. 3 shows an example embodiment of the method 100 with the mechanical ventilation training program. A training algorithm for the mechanical ventilation training program is stored in the non-transitory computer readable medium 15 of the mechanical ventilator 2. The training algorithm receives, as an input, the respiratory muscle pressure Pₘᵤₛ and/or the diaphragm thickness metric TFdi, and in some examples, information from the patient P (e.g., air pressure, air flow, diaphragm electrical activity (Edi), electromyography, auxiliary muscle activities, or US images from the US imaging device 18), and then outputs an automatic training program (shown in block 30 of FIG. 3), and corresponding control settings for the mechanical ventilator 2. Results from the mechanical ventilation training program can be displayed on the display device 14 as a graph or a dashboard.

The training program can be started or initiated when atrophy is detected or predicted (i.e., when the diaphragm thickness metric TFdi is decreasing), for example with manual ultrasound and/or when the patient P fails a first spontaneous breathing test (SBT), indicating a difficult weaning patient. The medical professional (i.e., a respiratory technician (RT)) starts the training program by activating this functionality in a user interface of the display device 14 of the mechanical ventilator 2 (i.e., a start button). The manual or wearable ultrasound transducer 20 can also work in the background and alarm the RT that the level of ventilation support for that particular patient is inadequate, too high, or too low (i.e., the diaphragm thickness metric TFdi outside of the safe range (i.e., 15-30%)) at any time during the ventilation.

The diaphragm thickness TFdi during a SBT is taken as a reference to determine the muscle function at the start of the training program (i.e., this includes the mental state of the patient P and the level of sedation). A level of support K is adapted until the initial TFdi is in a safe range to start with, for example between TFdi=15-30%.

The level of support K can be adapted to do a training program a couple of times a day. For example, two or three times a day (i.e., every 8-12 hours), K can be decreased with X% depending on the results of the titration. Optionally the number and duration of the intervals can be increased manually or automatically on a day-to-day basis depending on the patient's response. For example, the electronic controller 13 detects a decrease in the diaphragm thickness TFdi or the mean thickness; hence, it suggests increasing the duration or the number of training intervals depending on the patient response.

The daily average TFdi or daily average mean thickness d can be determined to inform the RT if the training program works or does not work (i.e., a muscle response “yes/no”), for program adaptation based on the muscle response (i.e., slow-down, or ramp-up depending on fatigue or strengthening), and for safety (i.e., the muscle does not respond or responds negatively). Information on the patient health status can be combined with the status of the diaphragm function.

The weekly average diaphragm thickening TFdi can be determined to represent the training effect (e.g., the recovery from atrophy, or to mark the end of the training program, after which the patient can be successfully extubated). Some target options for the training algorithm can include for example, a pre-determined mean diaphragm muscle thickening TFdi (for example the thickening when the patient was still healthy or when they were first admitted to the ICU prior to intubation is such data are available); a thickening range representative for similar healthy patients; a patient remains stable while K<50%, after which a SBT can confirm weaning success; a thickness and contractile activity reach a plateau (i.e., no further improvement is observed); and so forth. In case such targets are not set by the RT at the beginning of the training, the electronic controller 13 informs the RT when at least one of the pre-set targets is reached. The RT terminates the training program when the target is reached.

A user interface (UI) can be displayed on the display device 14 to help visualize the patient's status to the RT or the clinical team can be added. The average diaphragm thickening TFdi, and the P. are displayed in the UI, and the training portion of these data can be shown in different colors or shades. This information will be valuable to the clinical team in understanding better the patient progression.

In other embodiments disclosed herein, the diaphragm thickness metric calculation operation 104 includes: extracting one or more respiratory features of the patient P (e.g., a diaphragm thickness measured from the US images), comparing the extracted features with the biomechanical model 28, determining a respiratory muscle pressure Pₘᵤₛ from the comparing; and controlling the mechanical ventilator 2 to adjust one or more parameters of the mechanical ventilation therapy delivered to the patient P based on the determined respiratory muscle pressure Pₘᵤₛ. In some embodiments, when the respiratory muscle pressure Pₘᵤₛ falls below a predetermined respiratory muscle pressure threshold, the alert 26 can be output.

To generate the biomechanical model 28, the electronic controller 13 is configured to generate a patient geometry model 32 of the patient P from one or more images of the patient's exterior, and generating the biomechanical model 28 from the patient geometry model 32 and the ultrasound data.

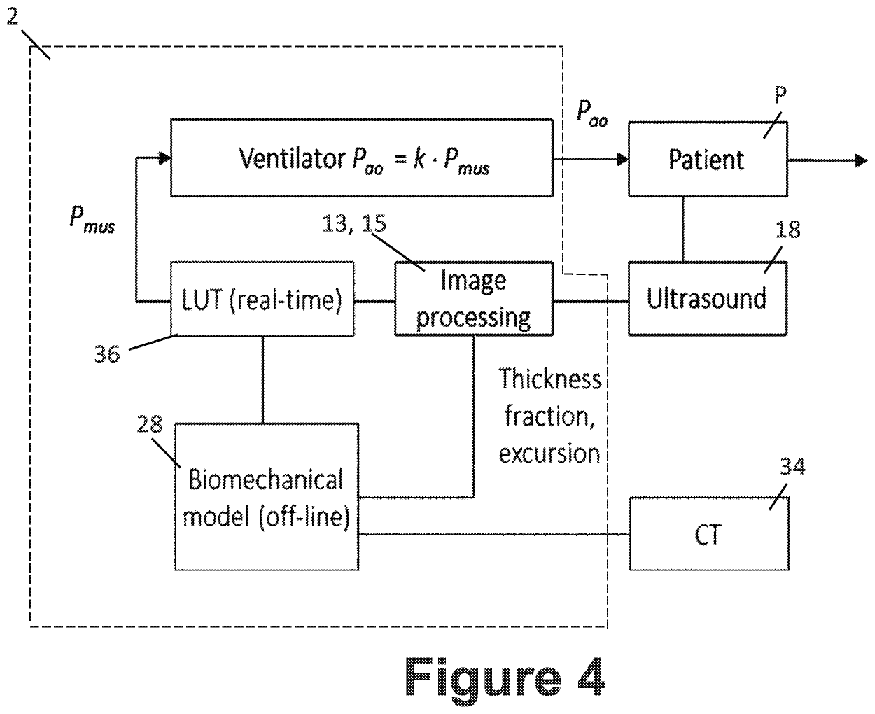

With continuing reference to FIGS. 1 and 2, FIG. 4 shows an example embodiment of the method 100 with the mechanical ventilation training program involving the biomechanical model 28. An additional imaging device (e.g., a CT imaging device 33 as shown in FIG. 1) acquires one or more CT images 34 of the patient P. It should be noted that the CT imaging device 33 may not be located in the same room, or even the same department, as the mechanical ventilator 2. For example, the CT imaging device 33 may be located in a radiology laboratory while the mechanical ventilator 2 may be located in an intensive care unit (ICU), cardiac care unit (CCU), in a hospital room assigned to the patient P, or so forth. This is diagrammatically indicated in FIG. 1 by separator line L.

Despite directly measuring the contractile activity of the diaphragm muscle, the thickening fraction TFdi is a surrogate measure for the pressure generated by the respiratory muscles Pmus. The respiratory muscle pressure Pₘᵤₛ depends on the patient specific geometry and mechanical properties of the thorax structures. It can be difficult to estimate the diaphragm strength directly from ultrasound measurements. Therefore, an option is to apply a patient specific biomechanical model 28 to calculate Pmus. The patient specific geometry can be obtained from the CT images 34 obtained at ICU admission. The muscle deformation is obtained from the ultrasound measurements. An advantage of the biomechanical model 28 is that the patient specific variables are considered in the estimation of Pₘᵤₛ. The generation of the biomechanical model 28 can be done off-line. The biomechanical model 28 simulates Pₘᵤₛ as a function of diaphragm thickening fraction and excursion. The model output Pₘᵤₛ is stored in a lookup table 36 in the non-transitory computer readable medium 15. The lookup table 36 is used in the closed loop system, as an intermediate step between the ultrasound image processing and the training program.

Safety is an important aspect of automated systems. A risk of proportional assist (PA) ventilation is that the system decreases the support if the diaphragm starts to weaken. In an extreme situation this means that both the respiratory effort and the support decrease to zero. Therefore, in some embodiments disclosed herein, a safety algorithm is needed to overwrite the electronic controller 13 if needed. The safety algorithm takes, as input, patient parameters such as SpO₂, EtCO₂, Ve (volume of gas exchange per minute), and/or the Pₘᵤₛ trajectory. If the patient P starts to respond with a negative trend, or if the muscle does not respond, the safety algorithm stops the training program. This is communicated in the user interface (“training aborted”, plus the reason for stopping) on the display device 16. The communication can take the form of the alert 26 (i.e., an alarm or warning). In some examples, a camera (not shown) can be added to assist the safety logic and alarming algorithm. During the training program, the RT or a member of the clinical team can remotely monitor the patient's progression using the additional information from the camera.

In some embodiments, the electronic controller 13 can be used to measure arousals when the patient P sleeps (such as EEG, vital signs, respiratory variability, bioimpedance, and so forth). Arousal from sleep when the training program is activated can be an indication that the level of support is too low. Since the patient P is already hooked up to the mechanical ventilator 2, it might be useful to measure their sleep state using parameters measured by the ventilator itself as opposed to adding additional sensors.

In other embodiments, a respiratory effort can also be measured, such as with a belt worn around the thorax, with a microphone positioned on the suprasternal notch, with intra-costal surface electromyography (EMG), or with an accelerometer or other acceleration, displacement or movement sensor mounted on, or close to the thorax—e.g., on the bed, under the mattress, etc. (none of which are shown in the FIGURES).

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A mechanical ventilation device comprising at least one electronic controller configured to:
receive ultrasound data related to a thickness of a diaphragm of a patient during inspiration and expiration while the patient undergoes mechanical ventilation therapy with a mechanical ventilator;
calculate a diaphragm thickness metric based on at least the ultrasound data; and
when the calculated diaphragm thickness metric does not satisfy an acceptance criterion, at least one of:
output an alert indicative of the calculated diaphragm thickness metric failing to satisfy the acceptance criterion; and
output a recommended adjustment to one or more parameters of the mechanical ventilation therapy delivered to the patient.

2. The device of claim 1, wherein the diaphragm thickness metric includes a diaphragm thickening ratio indicative of a diaphragm thickness during inspiration relative to a diaphragm thickness during expiration.

3. The device of claim 1, wherein the diaphragm thickness metric includes a mean diaphragm thickness over a respiratory cycle.

4. The device of claim 1, further comprising:
a wearable ultrasound transducer from which the at least one electronic controller receives the ultrasound data.

5. The device of claim 1, wherein the at least one electronic controller is configured to:
control the mechanical ventilator to adjust one or more parameters of the mechanical ventilation therapy delivered to the patient; and
iteratively repeat the receive, calculate, and control operations to provide feedback control of the mechanical ventilator based at least on whether the calculated diaphragm thickness metric satisfies the acceptance criterion.

6. The device of claim 1, further comprising:
an ultrasound imaging device configured to generate the ultrasound data;
wherein the at least one electronic controller is implemented in the ultrasound imaging device.

7. The device claim 1, wherein the electronic controller is configured to:
calculate the diaphragm thickness metric from the ultrasound data by:
extracting one or more respiratory features of the patient;
comparing the extracted features with a biomechanical model; and
determining a respiratory muscle pressure from the comparing; and
control the mechanical ventilator to adjust one or more parameters of the mechanical ventilation therapy delivered to the patient based on the determined respiratory muscle pressure.

8. The device of claim 7, wherein the electronic controller is configured to generate the biomechanical model by:
generating a patient geometry model of the patient from one or more images of the patient's exterior; and
generating the biomechanical model from the patient geometry model and the ultrasound data.

9. The device of claim 1, further including:
the mechanical ventilator; and
a second electronic controller implemented in the mechanical ventilator.

10. The device of claim 9, wherein the mechanical ventilation therapy delivered to the patient comprises a mechanical ventilation training program, and the second electronic controller is configured to:
detect when the calculated diaphragm thickness metric does not satisfy the acceptance criterion; and
adjust the mechanical ventilation training program until the calculated diaphragm thickness metric satisfies the acceptance criterion.

11. The device of claim 10, wherein the calculated diaphragm thickness metric comprises a respiratory muscle pressure (Pmus) calculated from the ultrasound data and a biomechanical model, and the second electronic controller is configured to adjust the mechanical ventilation training program until the calculated respiratory muscle pressure satisfies the acceptance criterion by:
adjusting a level-of-support parameter of the mechanical ventilation training program.

12. The device of claim 11, wherein the second electronic controller is configured to:
multiply the level-of-support parameter by the respiratory muscle pressure to determine an airway ventilation pressure value; and
continuously perform the mechanical ventilation training program until the airway ventilation pressure value falls below a predetermined training program threshold.

13. The device of claim 9, wherein the second electronic controller is configured to:
output an alert on a display device of the mechanical ventilator, the alert being indicative of the calculated diaphragm thickness metric failing to satisfy the acceptance criterion.

14. The device of claim 1, wherein the at least one electronic controller configured to is configured to:
control the mechanical ventilator to adjust one or more parameters of the mechanical ventilation therapy delivered to the patient.

15. A mechanical ventilation method comprising, with at least one electronic controller:
receiving ultrasound data related to a thickness of a diaphragm of patient during inspiration and expiration while the patient undergoes mechanical ventilation therapy with a mechanical ventilator;
calculating a diaphragm thickness metric based on at least the ultrasound data; and
when the calculated diaphragm thickness metric does not satisfy an acceptance criterion, at least one of:
outputting an alert indicative of the calculated diaphragm thickness metric failing to satisfy the acceptance criterion; and
outputting a recommended adjustment to one or more parameters of the mechanical ventilation therapy delivered to the patient.
